(19) United States
(12) Patent Application Publication
(10) Pub. No.: US 2023/0321395 A1
(43) Pub. Date: Oct. 12, 2023

(54) A DIAGNOSTIC KIT AND METHOD THEREFORE

(71) Applicant: PROSTALUND AB, LUND (SE)

(72) Inventors: Sonny SCHELIN, ROCKNEBY (SE); Anette ISRAELSSON, LUND (SE); Johan WENNERHOLM, LOMMA (SE)

(21) Appl. No.: 18/024,812

(22) PCT Filed: Sep. 28, 2021
(86) PCT No.: PCT/SE2021/050946

§ 371 Date: Mar. 6, 2023

(30) Foreign Application Priority Data
Oct. 1, 2020 (SE) 2051143-2

(51) Int. Cl. A61M 25/00; A61M 25/04; A61M 25/10; A61B 5/20; A61B 5/00

(52) U.S. Cl. CPC A61M 25/0017; A61M 25/04; A61M 25/10; A61B 5/204; A61B 5/6853; A61M 2210/1085

(57) ABSTRACT

The present invention describes a diagnostic kit comprising an indwelling catheter, a set volume of a saline solution (or corresponding solution) contained in a syringe and a drainage and diagnosis container with volume markings. Moreover, the present invention also refers to a method for performing a diagnostic test before a potential prostate surgery/treatment.

### FIELD OF THE INVENTION

The present invention relates to a diagnostic kit and method intended for diagnosis of a patient before a potential prostate surgery or treatment, such as e.g. an invasive prostate therapy.

### SUMMARY OF THE INVENTION

The present invention refers to a diagnostic kit comprising
an indwelling catheter comprising two connectable and separable main units being
an introduction stent unit with a top and bottom portion, said introduction stent unit also comprising a bladder fluid opening, wherein said introduction stent unit has a length possible to arrange with the top inside of the bladder of a patient and with the bottom portion arranged inside of the urethra before the external urinary sphincter muscle of the patient when seen from the bladder and outwards;
a catheter filling unit with an open bottom end and a top portion, said catheter filling unit being detachable form the indwelling catheter;
   said indwelling catheter also comprising
a balloon stopper unit in connection with and thus possible to fill via a balloon filling tube and out from a balloon fluid inlet opening arranged on the introduction stent unit, wherein the filling of the balloon stopper unit with a sterile fluid, preferably sterile water, is intended when the balloon stopper unit is arranged inside of the bladder of the patient to provide for a stopper function of the balloon stopper unit against the bladder neck of the patient; and
a pull thread fixedly arranged on the introduction stent unit to enable pulling of the introduction stent unit when the catheter filling unit is detached from the indwelling catheter;
   said diagnostic kit also comprising
a set volume of a saline solution (or corresponding solution) contained in a syringe connectable to the open bottom end of the catheter filling unit; and
a drainage and diagnosis container with volume markings.

Moreover, the present invention also refers to a method for performing a diagnostic test before a potential prostate surgery/treatment, said method comprising:
arranging an indwelling catheter inside of the urethra and up into the bladder of a patient,
   said indwelling catheter comprising two connectable and separable main units being
      an introduction stent unit with a top and bottom portion, said introduction stent unit also comprising a bladder fluid opening, wherein said introduction stent unit has a length possible to arrange with the top inside of the bladder of a patient and with the bottom portion arranged inside of the urethra before the external urinary sphincter muscle of the patient when seen from the bladder and outwards;
      a catheter filling unit with an open bottom end and a top portion, said catheter filling unit being detachable form the indwelling catheter;
         said indwelling catheter also comprising
a balloon stopper unit in connection with and thus possible to fill via a balloon filling tube and out from a balloon fluid inlet opening arranged on the introduction stent unit, wherein the filling of the balloon stopper unit is intended when the balloon stopper unit is arranged inside of the bladder of the patient to provide for a stopper function of the balloon stopper unit against the bladder neck of the patient; and
a pull thread fixedly arranged on the introduction stent unit to enable pulling of the introduction stent unit when the catheter filling unit is detached from the indwelling catheter;
   said method also comprising
filling the balloon stopper unit with a sterile fluid, preferably sterile water, to fixedly arrange the top of the introduction stent unit inside of the bladder of the patient;
flowing a volume of saline solution (or corresponding solution) contained in a syringe being connected to the open bottom end of the catheter filling unit into the bladder of the patient, preferably until the patient is in need or urinating;
noting the volume filled into the bladder;
detaching the catheter filling unit from the indwelling catheter;
instructing the patient to perform certain movements during a certain time frame;
organizing for patient-driven urination from the patient into a drainage and diagnosis container with volume markings;
noting the urinated volume from the patient; and finally
performing a diagnosis based on the calculation of urinated volume in relation to the volume of saline solution (or corresponding solution) flown into the bladder of the patient.

### SPECIFIC EMBODIMENTS OF THE INVENTION

Some specific embodiments of the present invention are provided below and in the attached claims. This is both valid for the diagnostic kit and for the method and use according to the present invention.

Moreover, below there is presented one alternative of an indwelling catheter for a diagnostic kit according to one specific embodiment of the present invention.

This alternative comprises an indwelling catheter 1 comprising: an introduction stent unit 2 with a top 2a and bottom portion 2b, and a bladder fluid opening 20;
a catheter filling unit 3 with an open bottom end 3a and a top portion 3b;
a balloon stopper unit 4 in connection with a balloon filling tube 5;
a balloon fluid inlet opening 21 arranged on the introduction stent unit 2;
the balloon filling tube (5) has a portion (50) with a spiral shape along the balloon filling tube (5) at a position outside and a distance from the bottom portion (2b) of the introduction stent unit (2), to enable anchoring inside of the urethra and outside of the external urinary sphincter muscle of the patient when seen from the bladder and outwards;
a receptacle 51 situated on the end of the balloon filling tube 5 before a valve arrangement 52;
and
a pull thread 6 fixedly arranged on the introduction stent unit 2.

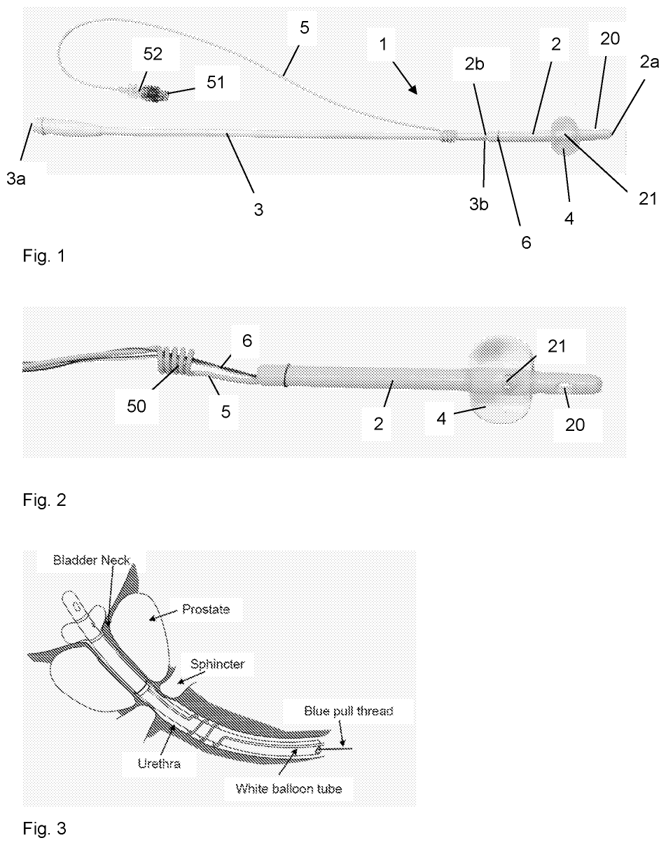

One embodiment thereof is presented in FIGS. 1-2.

In relation to the above it should be noted that the diagnostic kit according to the present invention also comprises a set volume of a saline solution (or corresponding solution) contained in a syringe connectable to the open bottom end of the catheter filling unit; and a drainage and diagnosis container with volume markings, which both are not shown above. For these two units, different shapes and sizes etc. may be used according to the present invention.

Moreover, according to one specific embodiment, the balloon filling tube has a portion with a spiral shape along the balloon filling tube at a position outside and a distance from the bottom portion of the introduction stent unit, to enable anchoring inside of the urethra and outside of the external urinary sphincter muscle of the patient when seen from the bladder and outwards. One alternative therefore is shown above. This anchoring ensures to hold the stent unit in position so this does not enter up into the bladder too much. In the same way, the balloon unit ensures that the stent unit is not pushed out from the position in the bladder.

Furthermore, and in reference to the figures above, according to one specific embodiment, the diagnostic kit also comprises a balloon filling syringe connectable to the balloon filling tube, preferably connectable to a receptacle situated on the end of the balloon filling tube, more preferably connectable to a receptacle situated on the end of the balloon filling tube before a valve arrangement, preferably wherein said balloon filling syringe comprises sterile fluid, suitably sterile water, intended to fill the balloon stopper unit.

Moreover, according to the present invention the optimal length of the introduction stent unit is in the range of 30-70 mm, from the top to the bottom portion of the introduction stent unit.

As should be understood from above, the present invention is also directed to a diagnostic method. According to one specific embodiment, the diagnosis is directed to evaluating if a patient is a suitable candidate for prostate surgery or treatment based on the calculation of urinated volume/volume of saline solution (or corresponding solution) flown into the bladder.

Furthermore, according to yet another embodiment, the diagnosis is directed to evaluating if a patient is a suitable candidate for prostate surgery or treatment based on the urinated volume during a set time range, preferably a time range of 30 seconds or less, more preferably a time range of 15 seconds.

Moreover, according to yet another embodiment of the present invention, the diagnosis also include at least one of the following:
evaluation with reference to if the patient is able to urinate on request;
evaluation with reference to if the patient is able to avoid leakage during movement.

In relation to the diagnostic method according to the present invention, the following criteria are such showing a strong result indicating that the patient is suitable for a prostate surgery/treatment:
the patient is able to urinate on request (this also implies the ability of stopping the urination on a request and starting the same again thereafter);
the patient is able to avoid leakage during movement;
the patient is able to urinate at least 1 dl during a time frame of less than 15 seconds; and
the patient is able to urinate at least 50% of the saline solution flown into the bladder.

The diagnostic method according to the present invention also provides insight on the patient's coordination ability of the bladder and the outer sphincter muscle, i.e. the patient's ability to avoid urination when the bladder is full and using the outer sphincter muscle to ensure this.

Moreover, the present invention is also directed to use of a diagnostic kit according to the present invention, for performing a diagnosis directed to evaluating if a patient is a suitable candidate for prostate surgery/treatment or not based on the calculation of urinated volume/volume of saline solution (or corresponding solution) flown into the bladder after performing filling a volume of saline solution (or corresponding solution) into the bladder of the patient and measuring the volume of urine from patient-driven urination after detachment of the catheter filling unit from the indwelling catheter.

Furthermore, the present invention also refers to use of a diagnostic kit according to the present invention, for performing a method according to the present invention, both as described above and in the claims.

Below there is presented more background and figures in relation to the diagnostic kit with an indwelling catheter according to the present invention. Furthermore, there is also presented more information in relation to the diagnostic method according to the present invention.

Firstly, stent during insertion is presented in FIG. 3

Insertion may be performed in accordance with the following
Apply anesthetic gel
Insert
Fill balloon, tie knots on balloon tube, cut off the valve
Empty the bladder
Fill the bladder to prepare for voiding tests
Separate rear part

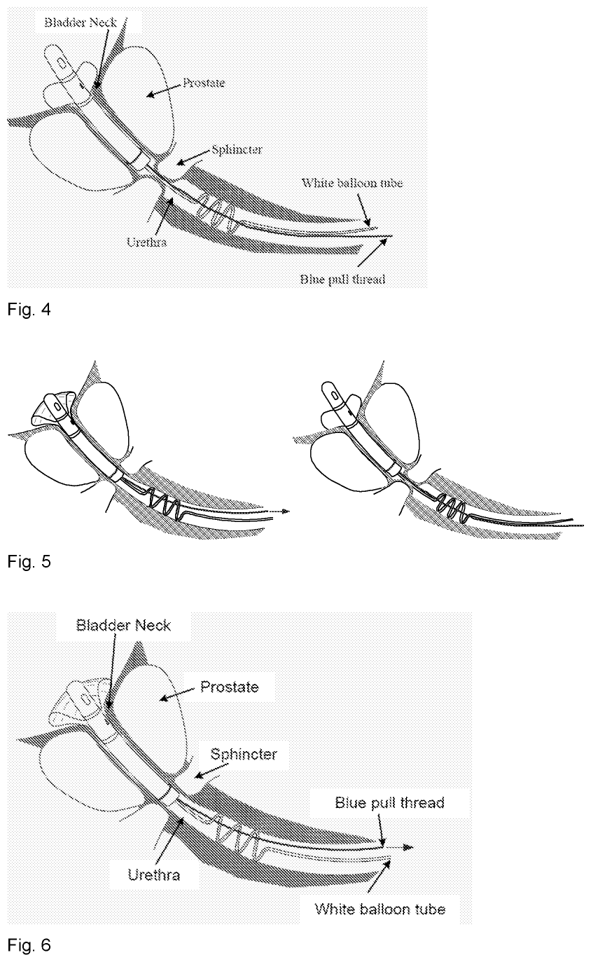

Secondly, self-controlled voiding is presented below in FIGS. 4-5
Open and close sphincter—physician (see FIG. 5)
Open and close sphincter—patient (see FIG. 5)

Moreover, self-catheterization is presented in FIG. 6

### Some Functionality of the Present Invention

Before separation the device functions as an ordinary “Foley” Catheter. Pull thread locks front and rear section together.
Relieves obstruction in the prostatic urethra.
Balloon and anchoring device holds stent in place,
Migrated stent, that may cause retention, can be repositioned by the patient himself.
Micturation can be patient-controlled using the “pull thread”.

As may be understood from above, the diagnostic kit according to the present invention has multi-functionality. The diagnostic kit provides for the following capabilities:
catheter function;
stent function;
diagnostic kit test (urodynamic test) as described above;
self-catheterization;

With reference to the diagnostic test capability, this may provide for:
Simulates removal of obstruction;
Provides an answer to if the patient benefit from an invasive therapy, with answers to questions such as: Is he incontinent?/Does he have normal bladder function?

1. A diagnostic kit comprising
an indwelling catheter comprising two connectable and separable main units being
an introduction stent unit with a top and bottom portion, said introduction stent unit also comprising a bladder fluid opening wherein said introduction stent unit has a length possible to arrange with the top inside of the bladder of a patient and with the bottom portion inside of the urethra before the external urinary sphincter muscle of the patient when seen from the bladder and outwards;
a catheter filling unit with an open bottom end and a top portion, said catheter filling unit being detachable form the indwelling catheter;
said indwelling catheter also comprising
a balloon stopper unit in connection with and thus possible to fill via a balloon filling tube out from a balloon fluid inlet opening arranged on the introduction stent unit, wherein the filling of the balloon stopper unit with a sterile fluid, preferably sterile water, is intended when the balloon stopper unit is arranged inside of the bladder of the patient to provide for a stopper function of the balloon stopper unit against the bladder neck of the patient; and
a pull thread fixedly arranged on the introduction stent unit to enable pulling of the introduction stent unit when the catheter filling unit is detached from the indwelling catheter;
said diagnostic kit also comprising
a set volume of saline solution, or a corresponding solution, contained in a syringe connectable to the open bottom end of the catheter filling unit; and
a drainage and diagnosis container with volume markings.

2. The diagnostic kit according to claim 1, wherein the balloon filling tube has a portion with a spiral shape along the balloon filling tube at a position outside and a distance from the bottom portion of the introduction stent unit, to enable anchoring inside of the urethra and outside of the external urinary sphincter muscle of the patient when seen from the bladder and outwards.

3. The diagnostic kit according to claim 1, also comprising a balloon filling syringe connectable to the balloon filling tube, preferably connectable to a receptacle situated on the end of the balloon filling tube, more preferably connectable to a receptacle situated on the end of the balloon filling tube before a valve arrangement, preferably wherein said balloon filling syringe comprises sterile fluid, preferably sterile water, intended to fill the balloon stopper unit.

4. The diagnostic kit according to claim 1, wherein the introduction stent unit from the top to the bottom portion has a total length in the range of 30-70 mm.

5. A method for performing a diagnostic test before a potential prostate surgery/treatment, said method comprising:
arranging an indwelling catheter inside of the urethra and up into the bladder of a patient,
said indwelling catheter comprising two connectable and separable main units being
an introduction stent unit with a top and bottom portion, said introduction stent unit also comprising a bladder fluid opening, wherein said introduction stent unit has a length possible to arrange with the top inside of the bladder of a patient and with the bottom portion arranged inside of the urethra before the external urinary sphincter muscle of the patient when seen from the bladder and outwards;
a catheter filling unit with an open bottom end and a top portion, said catheter filling unit being detachable form the indwelling catheter;
said indwelling catheter also comprising
a balloon stopper unit in connection with and thus possible to fill via a balloon filling tube and out from a balloon fluid inlet opening arranged on the introduction stent unit, wherein the filling of the balloon stopper unit is intended when the balloon stopper unit is arranged inside of the bladder of the patient to provide for a stopper function of the balloon stopper unit against the bladder neck of the patient; and
a pull thread fixedly arranged on the introduction stent unit to enable pulling of the introduction stent unit when the catheter filling unit is detached from the indwelling catheter;
said method also comprising
filling the balloon stopper unit with a sterile fluid, preferably sterile water, to fixedly arrange the top of the introduction stent unit inside of the bladder of the patient;
flowing a volume of saline solution, or a corresponding solution, contained in a syringe being connected to the open bottom end of the catheter filling unit into the bladder of the patient, preferably until the patient is in need or urinating;
noting the volume filled into the bladder;
detaching the catheter filling unit from the indwelling catheter;
instructing the patient to perform certain movements during a certain time frame;
organizing for patient-driven urination from the patient into a drainage and diagnosis container with volume markings;
noting the urinated volume from the patient; and finally
performing a diagnosis based on the calculation of urinated volume in relation to the volume of saline solution, or corresponding solution, flown into the bladder of the patient.

6. The method according to claim 5, wherein the diagnosis is directed to evaluating if a patient is a suitable candidate for prostate surgery or treatment based on the calculation of urinated volume/volume of saline solution, or corresponding solution, flown into the bladder.

7. The method according to claim 5, wherein the diagnosis is directed to evaluating if a patient is a suitable candidate for prostate surgery or treatment based on the urinated volume during a set time range, preferably a time range of 30 seconds or less, more preferably a time range of 15 seconds.

8. The method according to claim 5, wherein the diagnosis also include at least one of the following:
evaluation with reference to if the patient is able to urinate on request; and
evaluation with reference to if the patient is able to avoid leakage during movement.

9. A method comprising using a diagnostic kit according to claim 1, for performing a diagnosis directed to evaluating if a patient is a suitable candidate for prostate surgery/treatment or not based on the calculation of urinated volume/volume of saline solution, or corresponding solution, flown into the bladder after performing filling a volume of saline solution, or corresponding solution, into the bladder of the patient and measuring the volume of urine from patient-driven urination after detachment of the catheter filling unit from the indwelling catheter.

10. A method comprising using a diagnostic kit according to claim 1, for performing a method comprising:
arranging the indwelling catheter inside of the urethra and up into the bladder of a patient;
filling the balloon stopper unit with a sterile fluid, preferably sterile water, to fixedly arrange the top of the introduction stent unit inside of the bladder of the patient;
flowing a volume of saline solution, or a corresponding solution, contained in a syringe being connected to the open bottom end of the catheter filling unit into the bladder of the patient, preferably until the patient is in need or urinating;
noting the volume filled into the bladder;
detaching the catheter filling unit from the indwelling catheter;
instructing the patient to perform certain movements during a certain time frame;
organizing for patient-driven urination from the patient into a drainage and diagnosis container with volume markings;
noting the urinated volume from the patient; and
performing a diagnosis based on the calculation of urinated volume in relation to the volume of saline solution, or corresponding solution, flown into the bladder of the patient.
